(19) United States
(12) Patent Application Publication
(10) Pub. No.: US 2010/0048957 A1
(43) Pub. Date: Feb. 25, 2010

(54) METHOD TO PREPARE PURE CURCUMIN

(76) Inventor: Darrick S.H.L. Kim, Schaumburg, IL (US)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/303,602

(22) PCT Filed: Jun. 5, 2007
(86) PCT No.: PCT/US07/70379

§ 371 Date: Apr. 1, 2009

Related U.S. Application Data
(60) Provisional application No. 60/811,041, filed on Jun. 5, 2006.

(51) Int. Cl. C07C 45/78

(52) U.S. Cl. 568/324

(57) ABSTRACT

This invention describes a preparation of at least 99% by weight pure curcumin from less pure grades of curcumin utilizing phenol protecting groups to favor a selective recrystallization of curcumin in the presence of demethoxycurcumin and bis-demethoxycurcumin and other curcuminoids of minor composition.

### CROSS REFERENCE TO RELATED APPLICATIONS

This application claims the benefit of U.S. Provisional Application Ser. No. 60/811,041, filed Jun. 5, 2006, which is incorporated herein in its entirety by reference.

### FIELD OF INVENTION

The invention relates to a highly efficient approach to prepare at least 99% pure curcumin from technical grade (typically about 70% by weight) curcumin. This invention focuses on the difference in reactivity between 4-phenolic groups with or without the presence of methoxy group on its adjacent position. This invention relates to readily available phenol protecting groups to guide a selective recrystallization of curcumin in the presence of other curcuminoids of similar physical properties. This invention further relates to preparation of at least 99% pure curcumin, which may be suitable for pharmaceutical use.

### BACKGROUND

Curcumin (1) is a natural product isolated in abundance from Curcuma species, in particular, Curcuma longa (Zingiberaceae) Linn. See Park and Kim, J. Nat. Prod. 65: 1227 (2002) and U.S. Pat. No. 5,861,415. Recently, it has attracted considerable attention due to its antioxidant (Kawanishi et al., Antioxid. Redox Signal. 7: 1728 (2005)), antiinflamatory (Chianani-Wu, J. Altern. Complement Med. 9: 161 (2003)), antiviral (Vajragupta et al., Bioorg. Med. Chem. Lett. 15: 3364 (2005) and Ranjan et al., J. Sur. Res. 87: 1 (1999)), antifungal (Kim et al., J. Agric. Food Chem. 51: 1578 (2003)), antibacterial (Foryst-Ludwig et al., Biochem. Biophys. Res. Commun. 316: 1065 (2004)), anticancer (Lee et al., Antioxid. Redox Signal. 7: 1612 (2005)), chemopreventive activities (Duvoix et al., Cancer Lett. 223: 181 (2005)), and in particular, its ability to protect neuronal cells from beta-amyloid insult (Park and Kim, J. Nat. Prod. 65: 1227 (2002) and Yang et al., J. Biol. Chem. 280, 5892 (2005)), suggesting a potentially important drug candidate to prevent and/or treat Alzheimer's Disease.

Although curcumin (1) can be readily synthesized (Nurfina et al., Eur. J. Med. Chem. 32: 321 (1997)), isolation from the natural resources using recrystallization techniques remains the most economically viable supply method (U.S. Pat. No. 5,861,415), however with notable challenges. Curcumin (1) is typically prepared in about 70% purity as a mixture of curcuminoids, such as demethoxycurcumin (2), bis-demethoxycurcumin (3), and other minor curcuminoids constituting the remaining composition. Because of the similar physical properties of these curcuminoids, large scale preparation of at least 99% pure curcumin using conventional chromatographic or recrystallization technique remains a challenge. It was envisioned a simple chemical modification of phenol groups on these curcuminoids using a phenol protecting group may alter the physiochemical properties of these curcuminoids to favor a selective recrystallization of curcumin (1) to achieve>99% purity.

A protecting group's chemoselectivity based on the difference in electrosteric environment around the reaction site may be a useful tool in separating a mixture of compounds with similar physical properties. Acetyl, THP (tetrahydropyran) ether, and TBDMS (t-butyldimethylsilyl) ether are representative examples of widely used phenol protecting groups. See Greene and Wuts, Protective Groups in Organic Synthesis, 2^{nd} ed. 1991. It was reasoned that reaction rate of these protecting groups on phenol functionality at a 4-position could be influenced by electrosteric environment attributed by methoxy group on an adjacent 3-position. Removal of phenol protecting groups under mild reaction conditions has been reported. See Greene and Wuts, Protective Groups in Organic Synthesis, 2^{nd} ed. 1991. Herein is disclosed a simple chemical modification method that yields at least 99% pure curcumin from a mixture of curcuminoids (1-3).

### SUMMARY OF INVENTION

The present invention relates to method of obtaining at least about 90% pure curcumin from technical grade (about 70% by weight) curcumin, which can be suitable for pharmaceutical use. The difference in reactivity between 4-phenolic groups with or without the presence of methoxy group on its adjacent position can be manipulated to allow for curcumin isolation from a mixture of curcumin and curcuminoids having similar properties. Thus, disclosed herein are methods of purifying curcumin using readily available reagents which provide phenol protecting groups and selectively recrystallizing curcumin in the presence of other curcuminoids of similar physical properties.

In one aspect, the disclosure provides a method of obtaining purified curcumin comprising the steps of (a) admixing impure curcumin comprising curcumin and curminoids and having a curcumin purity up to about 75% by weight, a reagent which provides a phenol protecting group, and an optional catalyst under condition sufficient to control reactivity of the hydroxyl groups of the curcuminoids and the reagent which provides a phenol protecting group to form a mixture of curcumin and at least one curcuminoid having at least one phenol protecting group; (b) crystallizing the mixture of step (a) in at least one organic solvent to form curcumin crystals having a curcumin purity of at least about 90% by weight. In some embodiments, the curcumin crystals have a purity of at least about 95%, at least about 96%, at least about 97%, at least about 98%, at least about 99%, or at least about 99.5% by weight. In various embodiments, the crystallization step can be repeated to increase the purity of the curcumin crystals.

Another aspect is to provide a method to obtain a purified curcumin composition having a purity of at least about 99% comprising the steps of: (1) obtaining an impure curcumin composition such as a commercially available curcumin composition having a purity of about 50 to about 70%; (2) dissolving the impure curcumin in an appropriate solvent, examples of which include, but are not limited to, DMF, acetonitrile, dichloromethane, ethylene dichloride, DMSO, acetone and the like; (3) adding an appropriate phenol protecting groups to the reaction at various temperature to control the reaction rate, such as from about −50° C. to reflux condition; (4) adding an appropriate catalyst such as pyridine p-toluenesulfonate (PPTS) or dimethylaminopyridine (DMAP) to assist the reaction; (5) quenching the reaction by adding water or appropriate solvent and/or reagent to the reaction mixture, (6) adding an appropriate organic solvent, examples including, but not limited to, ethyl acetate, dichloromethane, chloroform, ethylene dichloride, or ethers; (7) washing the said organic solution to remove water soluble substance from the organic layer and repeating the step as needed; (8) removing the said solvent under vacuum or steady stream of air; (9) dissolving the reaction residue in a minimum amount of appropriate hot organic solvent, examples including, but not limited to, methanol, ethanol, acetone, methyl ethyl ketone, isopropyl alcohol, acetic acid, water, ethyl acetate, ethylene dichloride, dichloromethane, or mixtures thereof; (10) slowly cooling the solution down to room temperature to initiate the recrystallization of curcumin, which can further be induced by placing the recrystallizing solution in a refrigerator and slowly evaporating the solvent to increase the yield of curcumin; (11) collecting the crystals using vacuum filtration; (12) rinsing the crystals with cold solvent and further drying the crystals; and (13) optionally repeating the recrystallization step to obtain curcumin with higher purity as needed. According to one aspect of the invention a purified curcumin composition having a purity of greater than 99% is obtained in the presence of other curcuminoids.

The invention further provides methods of obtaining a purified curcumin composition having a purity of at least 99% by weight, said method comprising the steps of utilizing phenol protecting groups to alter the chemophysical properties of other curcuminoids to favor the preparation of pure curcumin in the presence of other curcuminoids.

The impure or crude curcumin can comprise curcumin and other curcuminoids. Other curcuminoids can constitute demethoxycurcumin, bis-demethoxycurcumin, calebin-A, 1-hydroxy-1,7-bis(4-hydroxy-3-methoxyphenyl)-6-heptene-3,5-dione, 1,7-bis(4-hydroxyphenyl)-1-heptene-3,5-dione, 1,7-bis(4-hydroxyphenyl)-1,4,6-heptatrien-3-one, 1,5-bis(4-hydroxy-3-methoxyphenyl)-1,4-pentadien-3-one, and other curcuminoids of minor composition.

The invention provides methods of obtaining a purified curcumin composition having a purity of greater than 99% said method where phenol protecting groups are in a form of ethers selected from the following list but not limited to: methyl, methoxymethyl, methylthiomethyl, t-butylthiomethyl, (phenyldimethylsilyl)methoxymethyl, benzyloxymethyl, p-methoxybenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl)ethoxymethyl, tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydrothiopyranyl S,S-dioxido, 1-[(2-chloro-4-methyl)phenyl]-4-methoxypiperidin-4-yl, 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methyoxybenzyl, 3,4-dimethoxybenzyl, o-nitrobenzyl, p-nitrobenzyl, p-halobenzyl, 2,4-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,4″-tris(levulinoyloxyphenyl )methyl, 4,4′,4″-tris(benzoyloxyphenyl)methyl, 3-(imidazol-1-ylmethyl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, and the like.

According to one aspect of the invention a method of obtaining a purified curcumin composition having a purity of at least 99% by weight is provided where phenol protecting groups are in a form of a silyl ether. The silyl ether can be selected from: trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxyphenylsilyl, and the like.

Also provided by the invention is a method of obtaining a purified curcumin composition having a purity of at least 99% by weight said method where phenol protecting groups are in a form of esters selected from the following list but not limited to: formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, p-P-phenylacetate, 3-phenylpropionate, 4-oxopentanoate (levulinate), 4,4-(ethylenedithio)pentanoate, pivaloate, adamantoate, crotonate, 4-methoxycrotonate, benzoate, p-phenylbenzoate, 2,4,6-trimethylbenzoate (mesitoate), 2-iodobenzoate, 4-azidobutyrate, 4-nitro-4-methylpentanoate, o-(dibromomethyl)benzoate, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate (Tigloate), o-(methoxycarbonyl)benzoate, p-P-benzoate, α-naphthoate, dimethylphosphinothioyl, 2,4-dinitrophenylsulfenate, and the like.

Also provided by the invention is a method of obtaining a purified curcumin composition having a purity of at least 99% by weight said method where phenol protecting groups are in a form of carbonates selected from the following list but not limited to: methyl, 9-fluorenylmethyl, ethyl, 2,2,2-trichloroethyl, 2-(methylthiomethoxy)ethyl, 2-(trimethylsilyl)ethyl, 2-(phenylsulfonyl)ethyl, 2-(triphenylphosphonio)ethyl, isobutyl, vinyl, allyl, p-nitropheny, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate.

The invention also provides a method of obtaining a purified curcumin composition having a purity of at least 99% by weight said method where phenol protecting groups are in a form of sulfonates and others selected from the following list but not limited to: sulfate, methanesulfonate (mesylate), benzylsulfonate, tosylate, 2-formylbenzenesulfonate, nitrate, borate, alkyl N,N,N′,N′-tetramethylphosphorodiamidate, N-phenylcarbamate and the like.

The invention also provides a method of obtaining a purified curcumin composition having a purity of at least 99% by weight said method using recrystallization technique in the presence of other phenol protecting groups attached curcuminoids. According to one aspect of the invention the recrystallization solvent systems are selected from singly, or in a mixture of but not limited to: methanol, ethanol, acetone, methyl ethylketone, isopropyl alcohol, acetic acid, water, ethyl acetate, ethylene dichloride, dichloromethane, and the like. According to one aspect of the invention a method of obtaining a purified curcumin composition having a purity of at least 99% by weight is provided wherein said curcuminoids are dissolved in minimum amount of said solvent(s) at a temperature above room temperature. According to another aspect of the invention a method of obtaining a purified curcumin composition having a purity of at least 99% by weight is provided wherein the dissolved curcuminoids are slowly cooled to room temperature and slowly evaporating the solvent to induce recrystallization of curcumin. More preferably the recrystallizing curcuminoids are placed in a refrigerator to further assist the recrystallization to increase the yield of curcumin.

The purified curcumin composition having a purity of at least 99% by weight can be obtained wherein the curcumin crystals are collected by vacuum filtration or wherein the collected curcumin crystals are dried and collected.

### BRIEF DESCRIPTION OF THE DRAWINGS

FIG. 1 depicts the structures of various curcuminoids including curcumin (1); demethoxy curcumin (2); and bis-demethoxy curcumin (3); and

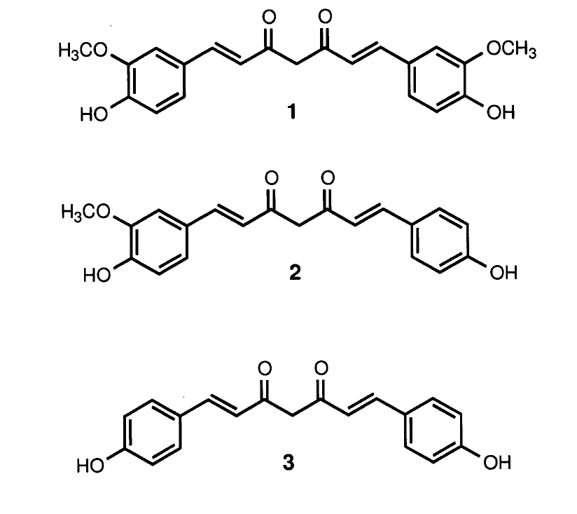

FIG. 2 depicts a scheme for the protection and deprotection of curcuminoids.

### DETAILED DESCRIPTION OF THE INVENTION

The present invention provides an improved strategy for obtaining highly purified (at least 90%, at least 95%, at least 96%, at least 97%, at least 98%, at least 99%, or at least 99.5% purified) curcumin compositions. In some cases, a phenol protecting group is used to modify a curcuminoid in the presence of curcumin to allow for purification of curcumin from the curcumin/curcuminoid mixture. Typically, the phenol protecting group is an ether, a silyl ether, a carbonate, or a sulfonate. In some specific cases, the phenol protecting group is a THP ether, acetyl, and/or TBDMS ether.

The methoxy group of the 3-position of a curcuminoid may influence the reaction rate of adjacent phenol group on 4-position such that curcuminoids 2 and 3 will predominantly acquire the protecting groups on their 4-phenol functionality, thus altering their physical properties to favor the crystallization of curcumin (1).

As used herein, the term “reagent which provides a phenol protecting group” is a reagent which reacts with a phenol (hydroxyl group on an aromatic moiety) to provide a protecting group. A protecting group is a moiety which masks the functional group being protect (here a phenol hydroxyl group) and which can then be removed to unmask the function group. Nonlimiting examples of protecting groups for phenols include ethers, esters, silyl ethers, carbonates, sulfonates, and the like. A reagent which provides a protecting group typically has reactive functionality which allows reaction between the reagent and the functional group to be protected. For example, a silyl chloride can be used to transform a phenol to a silyl ether. Other examples of reagents which provide a phenol protecting group can be found in Greene and Wuts, Protective Groups in Organic Synthesis, 2^{nd} ed. 1991.

Specific nonlimiting examples of protecting groups methyl, methoxymethyl, methylthiomethyl, t-butylthiomethyl, (phenyldimethylsilyl)methoxymethyl, benzyloxymethyl, p-methoxybenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl )ethoxymethyl, tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydrothiopyranyl S,S-dioxido, 1-[(2-chloro-4-methyl)phenyl]-4-methoxypiperidin-4-yl, 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methyoxybenzyl, 3,4-dimethoxybenzyl, o-nitrobenzyl, p-nitrobenzyl, p-halobenzyl, 2,4-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-ylmethyl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, and benzisothiazolyl S,S-dioxido, trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxyphenylsilyl, formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, p-P-phenylacetate, 3-phenylpropionate, 4-oxopentanoate (levulinate), 4,4-(ethylenedithio)pentanoate, pivaloate, adamantoate, crotonate, 4-methoxycrotonate, benzoate, p-phenylbenzoate, 2,4,6-trimethylbenzoate (mesitoate), 2-iodobenzoate, 4-azidobutyrate, 4-nitro-4-methylpentanoate, o-(dibromomethyl)benzoate, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate (Tigloate), o-(methoxycarbonyl)benzoate, p-P-benzoate, α-naphthoate, dimethylphosphinothioyl, 2,4-dinitrophenylsulfenate, methyl, 9-fluorenylmethyl, ethyl, 2,2,2-trichloroethyl, 2-(methylthiomethoxy)ethyl, 2-(trimethylsilyl)ethyl, 2-(phenylsulfonyl)ethyl, 2-(triphenylphosphonio)ethyl, isobutyl, vinyl, allyl, p-nitropheny, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate, sulfate, methanesulfonate (mesylate), benzylsulfonate, tosylate, 2-formylbenzenesulfonate, nitrate, borate, alkyl N,N,N′,N′-tetramethylphosphorodiamidate, and N-phenylcarbamate.

The conditions sufficient to control reactivity of the hydroxyl (phenol) groups of the curcumin and curcuminoids can be chosen by the person of skill in the art, in view of the present disclosure. The concentration of the reagents, use of an optional catalyst (such as DMAP or PPTS), temperature of the reaction, and/or reaction time can be selected to provide appropriate selectivity for a 4-hydroxyl next to a 3-methoxy group. The temperature of the reaction can be about −50° C. to reflux. Specific temperatures include, but are not limited to, about −50° C. to about 0° C., about 10° C. to about 25° C., about 30° C. to about 100° C., about 50° C. to about 90° C., about 50° C. to about 85° C., about 55° C. to about 80° C., or about 65° C. to about 75° C. In some cases, the temperature is above room temperature (typically about 25 to about 27° C.).

The organic solvent suitable for the crystallization step can be any solvent that is compatible with the curcumin, curcuminoid, and protected curcuminoid mixture. Typically, the organic solvent is methanol, ethanol, acetone, methyl ethylketone, isopropyl alcohol, acetic acid, water, ethyl acetate, ethylene dichloride, dichloromethane, or mixtures thereof. Other organic solvents contemplated include ethyl acetate, dichloromethane, chloroform, ethylene dichloride, or ethers. In some cases, the crystallization includes cooling the mixture to below room temperature to facilitate the formation of curcumin crystals. In specific cases, the temperature is less than about 15° C., less than about 10° C., less than about 4° C., less than about 0° C., less than about −10° C., or less than about −20° C.

In various cases, the crystals of curcumin can be collected. Collection of crystals can be via any known technique, and include via filtration. In some cases, the crystals are filtered and dried. Filtration can be via any known means, including gravity, vacuum, and the like.

### EXAMPLES

The present invention is further explained by the following examples which should not be construed by way of limiting the scope of the present invention.

### Example 1

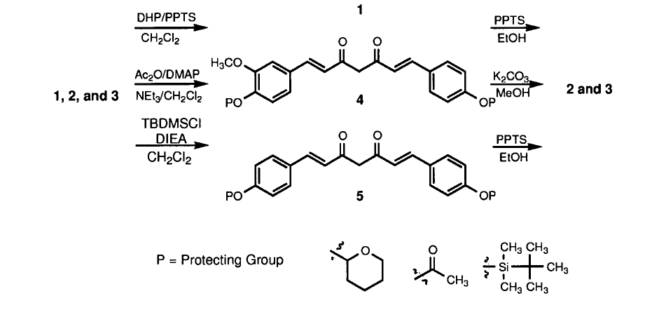

For this study 4:1:1 mixture of previously isolated curcuminoids 1, 2, and 3 were used. See Park and Kim, J. Nat. Prod. 65: 1227 (2002). Approximately 3.2 equivalent of protecting reagents were used to selectively capture the phenol functionality on 4-position without methoxy group at the adjacent position (Scheme 1 in FIG. 2).

The first batch of (4:1:1) curcuminoid mixture was reacted with dihydropyran (DHP) in dimethylformamide (DMF) in the presence of PPTS as catalyst overnight. See Greene and Wuts Protective Groups in Organic Synthesis, 2^{nd} ed. 1991. After aqueous work up, recrystallization of curcumin (1) was induced using hot acetone/H₂O system (2×) to afford >99% curcumin in 79% yield. The second batch of (4:1:1) curcuminoid mixture was reacted with acetic anhydride in triethylamine/DMF in the presence of DMAP as catalyst overnight. See Greene and Wuts Protective Groups in Organic Synthesis, 2^{nd} ed. 1991. Aqueous work up and recrystallization using hot acetone/H₂O system described above afforded >99% curcumin in 72% yield. The third batch of (4:1:1) curcuminoid mixture was reacted overnight with TBMDSCl in diisopropylethylamine/DMF. See Greene and Wuts Protective Groups in Organic Synthesis, 2^{nd} ed. 1991. Aqueous work up and recrystallization using hot acetone/H₂O system afforded >99% curcumin in 84% yield.

The remaining residues were subjected to protecting group removal condition without isolation: PPTS/ethanol system for THP ether, K₂CO₃/methanol system for acetyl, and 1,1,3,3-tetramethylguanidine(TMG)/acetonitrile (CH₃CN) system for TBDMS ether protecting groups (Oyama and Kondo, Org. Lett. 5: 209 (2003). The resulting residues were purified using column chromatography over silica gel to afford curcuminoids in various yields to confirm the efficacy of the method.

Of the THP ether protecting group work up, the overall yield of curcumin (1) was 87% while the overall curcuminoid yield was 88%. Of the acetyl protecting group work up, the overall yield of curcumin (1) was 90% and the overall curcuminoid yield also was 90%. Of the TBDMS protecting group work up, the overall yield of curcumin (1) was 88% while the overall curcuminoid yield was 90%. Table 1 shows curcuminoid yields from this procedure.

TABLE 1
Curcuminoid recovery yield from 4-phenol group protection,
recrystallization of curcumin (1), deprotection, and chromatographic
isolation using (4:1:1) curcuminoid mixture (600 mg)

| Protecting Group | Yield (%) | | | |
|---|---|---|---|---|
| Compounds | 1* | 2 | 3 | Overall yield (%) |
| THP | (79/87) | 90 | 89 | 88 |
| Acetyl | (72/90) | 91 | 88 | 90 |
| TBDMS | (84/88) | 93 | 90 | 89 |

*Note: The yields are presented as (recrystallization/overall).

### Example 2

Table 2 shows curcuminoid yields from processing of commercially available about 70% technical grade curcumin. The composition of about 70% technical grade curcumin was assumed to be a mixture of 70%, 20%, and 5%, curcuminoid 1, 2, and 3, respectively, and 5% unknown impurity based on a previous report (U.S. Pat. No. 5,861,415). The reactions were carried out as for Example 1. The results confirmed that this procedure is a simple and effective method to obtain >99% pure curcumin from a technical grade curcumin containing a mixture of curcuminoids that are similar in physiochemical properties.

Procedures using bulky protecting groups (THP ether and TBDMS ether) afforded higher curcumin (1) yield from recrystallization. The chromatographic isolation of products after removal of protecting groups and aqueous work up further confirmed the efficacy of this procedure. The overall curcuminoid yields remained comparable but were less than that of investigation using pure curcuminoids (Table 1), suggesting the influence of unidentified impurities in the technical grade. The purity of isolated curcuminoids was established based on their isolated yields, ¹HNMR spectral data, and mixed melting point analyses. Curcumin (1) mixed with about 2% curcuminoids 2 or 3 afforded melting point about 1° C. lower and wider range (about 1 to about 1.5° C.) than that of pure curcumin. All curcumin (1) obtained from recrystallization afforded sharp melting point of 183.5-184° C.

TABLE 2
Curcuminoid recovery yield from 4-phenol group protection,
recrystallization of curcumin (1), deprotection, and chromatographic
isolation using technical grade curcumin (1.0 g)

| Protecting Group | Yield (mg) | | | |
|---|---|---|---|---|
| Compounds | 1* | 2 | 3 | Overall yield (w/w %) |
| THP | (460/604) | 158 | 28 | 79 |
| Acetyl | (410/626) | 148 | 26 | 80 |
| TBDMS | (456/616) | 162 | 32 | 81 |

*Note: The yields are presented as (recrystallization/overall).

### Experimental Details for Examples 1 and 2

Materials and Instruments. All solvents and reagents were purchased from Aldrich and used without further purification. Compounds 1, 2, and 3 isolated from Curcuma longa were used. The ¹H NMR (300 MHz) and ¹³CNMR (75 MHz) spectra were measured in DMSO-d6 or acetone-d6 using Bruker 300 MHz spectrometer. The chemical shifts are reported in δ (ppm) relative to TMS. Melting points were determined in open capillary tubes with a Thomas-Hoover apparatus and were uncorrected.

A General Procedure for Preparation of THP ether. To an anhydrous DMF solution (100 mL) containing compounds (600 mg) 1, 2, and 3 (4:1:1) was added 3.1 equiv. anhydrous DHP and catalytic amount of PPTS (20 mg) at room temperature and stirred overnight under N₂ atmosphere.

A General Procedure for Preparation of Acetate. To an anhydrous DMF solution (100 mL) containing compounds (600 mg) 1, 2, and 3 (4:1:1) was added 3.5 equiv. anhydrous triethylamine, catalytic amount of DMAP (20 mg), and acetic anhydride (3.1 equiv.) at 0° C., warmed to room temperature, and stirred overnight under N₂ atmosphere.

A General Procedure for Preparation of Silyl ether. To an anhydrous DMF solution (100 mL) containing compounds (600 mg) 1, 2, and 3 (4:1:1) was added 3.5 equiv. anhydrous diisopropylethylamine, and TBDMSCl (3.1 equiv.) at room temperature, and stirred overnight under N₂ atmosphere.

A General Procedure for Aqueous Work up and Recrystallization of Curcumin (1). The reaction mixture was added H₂O (200 mL) and extracted with ethyl acetate (100 mL×4). The organic layers were combined, washed with H₂O (50 mL×3). The combined aqueous layer was extracted with ethyl acetate (50 mL×2). The organic layers were combined, dried (MgSO₄), filtered, and the solvent was removed under vacuum. The resulting residue was dissolved in minimum amount of hot acetone, dropwisely added H₂O to induce precipitation, reheated to re-dissolve, slowly cooled at room temperature and placed in a refrigerator to induce crystallization of curcumin (1). The crystals were filter collected dried, and recrystallized using acetone/H₂O system as before to afford >99% pure curcumin. The purity of curcumin was confirmed by ¹H NMR and mixed melting point comparison.

A General Procedure for Removing THP Group. To an ethanol solution (50 mL) containing the residue (200 mg) was added PPTS (20 mg) at room temperature, stirred overnight, added H₂O (20 mL), and the solvent was reduced to ˜50% volume under stream of air. To the residue was added ethyl acetate (100 mL), washed with H₂0 (50 mL×3). The aqueous layers were combined and washed with ethyl acetate (50 mL×2). The organic layers were combined, dried (MgSO₄), filtered, and the solvent was removed under vacuum. The residue was column chromatographed over silica gel using gradient ethylacetate/pet ether/isopropyl alcohol.

A General Procedure for Removing Acetyl Group. To a methanol solution (50 mL) containing the residue (200 mg) was added K₂CO₃ (20 mg) at room temperature, stirred overnight, added dropwise 10% AcOH to neutralize K₂CO₃, added H₂O (20 mL), and the solvent was reduced to ˜50% volume under stream of air. To the residue was added ethyl acetate (100 mL), washed with H₂O (50 mL×3). The aqueous layers were combined and washed with ethyl acetate (50 mL×2). The organic layers were combined, dried (MgSO₄), filtered, and the solvent was removed under vacuum. The residue was column chromatographed over silica gel using gradient ethylacetate/pet ether/isopropyl alcohol.

A General Procedure for Removing Silyl ether Group. To a CH₃CN solution (50 mL) containing the residue (200 mg) was added 1,1,3,3-tetramethylguanidine (100 mg), H₂O (0.5 mL) and stirred at 50° C. for two hours. The reaction mixture was cooled to room temperature, added H₂O (20 mL), and the solvent was reduced to ˜50% volume under stream of air. To the residue was added ethyl acetate (100 mL), washed with H₂O (50 mL×3). The aqueous layers were combined and washed with ethyl acetate (50 mL×2). The organic layers were combined, dried (MgSO₄), filtered, and the solvent was removed under vacuum. The residue was column chromatographed over silica gel using gradient ethylacetate/pet ether/isopropyl alcohol.

Curcumin 1

Yellow crystals; mp 183.5-184° C.; ¹H NMR (300 MHz, DMSO-d₆) δ7.56 (2H, d, J=15.8 Hz, H-1 and H-7), 7.32 (2H, d, J=1.5 Hz, H-2′ and H-2″), 7.15 (2H, dd, J=8.2 and 1.5 Hz, H-6′ and H-6″), 6.83 (2H, d, J=8.2 Hz, H-5′ and H-5″), 6.76 (2H, d, J=15.8 Hz, H-2 and H-6), 6.05 (1H, s, H-4), 3.84 (6H, s, OCH₃-3′ and OCH₃-3″) ppm; ¹³C NMR (75 MHz, DMSO-d₆) δ184.1 (C-3 and C-5), 150.2 (C-4′ and C-4″), 148.8 (C-3′ and C-3″), 141.6 (C-1 and C-7), 127.2 (C-1′ and C-1″), 124.0 (C-6′ and C-6″), 121.9 (C-2 and C-6), 116.6 (C-5′ and C-5″), 112.1 (C-2′ and C-2″), 101.8 (C-4), 56.5 (OCH₃-3′ and OCH₃-3″) ppm.

Demethoxycurcumin 2

Yellow powder; mp 181-182 ° C.; ¹H NMR (300 MHz, acetone-d6) δ7.62 (1H, d, J=15.3 Hz, H-7), 7.61 (1H, d, J=15.3 Hz, H-1), 7.57 (2H, d, J=8.1 Hz, H-2″ and H-6″), 7.35 (1H, d, J=1.8 Hz, H-2′), 7.18 (1H, dd, J=8.1 and 1.8 Hz, H-6′) 6.91 (2H, d, J=8.1 Hz, H-3″ and H-5″), 6.88 (1H, d, J=8.1 Hz, H-5′), 6.73 (1H, d, J=15.3 Hz, H-6), 6.67 (1H, d, J=15.3 Hz, H-2), 5.99 (1H, s, H-4), 3.93 (3H, s, OCH₃-3′) ppm; ¹³C NMR (75 MHz, acetone-d₆) δ188.4 (C-3 and C-5), 160.4 (C-4″), 150.0 (C-4′), 148.8 (C-3′), 141.2 (C-7), 141.0 (C-1), 130.8 (C-2″ and C-6″), 128.2 (C-1″), 127.8 (C-1′), 123.7 (C-6′), 122.4 (C-6), 122.1 (C-2), 116.8 (C-3″ and C-5″), 116.2 (C-5′), 111.7 (C-2′), 101.5 (C-4), 56.4 (OCH₃-3′) ppm.

Bisdemethoxycurcumin 3

Yellow needles; mp 232-233 ° C.; ¹H NMR (300 MHz, acetone-d₆) δ7.62 (2H, d, J=15.6Hz, H-1 and H-7),7.58 (4H, d, J=8.4 Hz, H-2′, H-2″, H-6′and H-6″), 6.91 (4H, d, J=8.4 Hz, H-3′, H-3″, H-5′ and H-5″), 6.68 (2H, d, J=15.6 Hz, H-2 and H-6), 5.99 (1H, s, H-4) ppm; ¹³C NMR (75 MHz, acetone-d₆), δ 184.0 (C-3 and C-5), 160.0 (C-4′ and C-4″), 140.8 (C-1 and C-7), 130.6 (C-2′, C-2″, C-6′ and C-6″), 126.9 (C-1′ and C-1″), 121.4 (C-2 and C-6), 116.5 (C-3′, C- 3″, C-5′ and C-5″), 101.6 (C-4) ppm.

### REFERENCES

Park, S.-Y.; Kim, D. S. H. L., Discovery of natural products from Curcuma longa that protect cells from beta-amyloid insult: a drug discovery effort against Alzheimer's disease. J. Nat. Prod. 2002, 65, 1227.
Majeed, M.; Badmaev, V.; Rajendran, R., Bioprotectant composition, method of use and extraction process of curcuminoids. 1999, U.S. Pat. No. 5,861,415.
Kawanishi, S.; Oikawa, S.; Murata, M., Evaluation for safety of antioxidant chemopreventive agents. Antioxid. Redox Signal. 2005, 7, 1728.
Chianani-Wu, N., Safety and anti-inflammatory activity of curcumin: a component of turmeric (Curcuma longa). J. Altern. Complement Med. 2003, 9, 161.

Vajragupta, O.; Boonchoong, P.; Morris, G. M.; Olson, A. J., Active site binding modes of curcumin in HIV-1 protease and integrase. Bioorg. Med. Chem. Lett. 2005, 15, 3364.
Ranjan, D.; Johnston, T. D.; Reddy, K. S.; Wu, G.; Bondada, S.; Chen, C., apoptosis mediates inhibition of EBV-transformed lymphoblastoid cell line proliferation by curcumin. Enhanced J. Sur. Res. 1999, 87, 1.
Kim, M.-K.; Choi, G.- J.; Lee, H.-S., Fungicidal property of Curcuma longa L. rhizome-derived curcumin against phytopathogenic fungi in a greenhouse. J. Agric. Food Chem. 2003, 51, 1578.
Foryst-Ludwig, A.; Neumann, M.; Schneider-Brachert, W.; Naumann, M., Curcumin blocks NF-kB and the motogenic response in Helicobacter pylori-infected epithelial cells. Biochem. Biophys. Res. Commun. 2004, 316, 1065.
Lee, K. W.; Kim, J.-H.; Lee, H. J.; Surh, Y.-J., Curcumin inhibits phorbol ester-induced up-regulation of cyclooxygenase-2 and matrix metalloproteinase-9 by blocking ERK1/2 phosphorylation and NF-kB transcriptional activity in MCF10A human breast epithelial cells. Antioxid. Redox Signal. 2005, 7, 1612.
Duvoix, A.; Blasius, R.; Delhalle, S.; Schnekenburger, M.; Morceau, F.; Henry, E.; Dicato, M.; Diederich, M., Chemopreventive and therapeutic effects of curcumin. Cancer Lett. 2005, 223, 181.
Yang, F.; Lim, G. P.; Begum, A. N.; Ubeda, O. J.; Simmons. M. R.; Ambegaokar, S. S.; Chen, P.; Kayed, R.; Glabe, C. G.; Frautschy, S. A.; Cole, G. M., Curcumin inhibits formation of amyloid b oligomers and fibrils, binds plaques, and reduces amyloid in vivo. J. Biol. Chem. 2005, 280, 5892.
Nurfina, A. N.; Reksohadiprodjo, M. S.; Timmerman, H.; Jenie, U. A.; Sugiyanto, D.; van der Goot, H., Synthesis of some symmetrical curcumin derivatives and their anti-inflammatory activity. Eur. J. Med. Chem. 1997, 32, 321.
Greene, T. W.; Wuts, P. G. M. Protective Groups in Organic Synthesis, 2^{nd} ed. 1991, John Wiley & Sons, Inc. and references sited therein.
Oyama, K.; Kondo, T., A novel and convenient chemoselective deprotection method for both silyl and acetyl groups on acidic hydroxyl groups such as phenol and carboxylic acid by using a nitrogen organic base, 1,1,3,3-tetramethylguanidine. Org. Lett. 2003, 5, 209.

All patents, publications and references cited herein are hereby fully incorporated by reference. In case of conflict between the present disclosure and incorporated patents, publications and references, the present disclosure should control.

What is claimed is:

1. A method of obtaining purified curcumin comprising the steps of:
a) admixing (1) impure curcumin comprising curcumin and curcuminoids and having a curcumin purity up to about 75% by weight, (2) a reagent which provides a phenol protecting group, and (3) an optional catalyst under conditions sufficient to control reactivity of the hydroxyl groups of the curcuminoids and the reagent which provides a phenol protecting group to form a mixture of curcumin and at least one curcuminoid having at least one phenol protecting group;
b) crystallizing the mixture of step (a) in at least one organic solvent to form curcumin crystals, wherein the curcumin crystals have a curcumin purity of at least about 99% by weight.

2. The method of claim 1, wherein the pure curcumin has a purity of at least about 99.5% by weight.

3. The method of claim 1, wherein the curcuminoids comprise demethoxycurcumin, bis-demethoxycurcumin, calebin-A, 1-hydroxy-1,7-bis(4-hydroxy-3-methoxyphenyl)-6-heptene-3,5-dione, 1,7-bis(4-hydroxyphenyl)-1-heptene-3,5-dione, 1,7-bis(4-hydroxyphenyl)-1,4,6-heptatrien-3-one, 1,5-bis(4-hydroxy-3-methoxyphenyl)-1,4-pentadien-3-one, or mixtures thereof.

4. The method of claim 1, wherein the phenol protecting group is an ether.

5. The method of claim 4, wherein the ether is selected from the group consisting of methyl, methoxymethyl, methylthiomethyl, t-butylthiomethyl, (phenyldimethylsilyl)methoxymethyl, benzyloxymethyl, p-methoxybenzyloxymethyl, (4-methoxyphenoxy)methyl, guaiacolmethyl, t-butoxymethyl, 4-pentenyloxymethyl, siloxymethyl, 2-methoxyethoxymethyl, 2,2,2-trichloroethoxymethyl, bis(2-chloroethoxy)methyl, 2-(trimethylsilyl)ethoxymethyl, tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydrothiopyranyl S,S-dioxido, 1-[(2-chloro-4-methyl)phenyl]-4-methoxypiperidin-4-yl, 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methyoxybenzyl, 3,4-dimethoxybenzyl, o-nitrobenzyl, p-nitrobenzyl, p-halobenzyl, 2,4-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-ylmethyl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, and benzisothiazolyl S,S-dioxido.

6. The method of claim 1, wherein the phenol protecting group is a silyl ether.

7. The method of claim 6, wherein the silyl ether is selected from the group consisting of trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, and t-butylmethoxyphenylsilyl.

8. The method of claim 1, wherein the phenol protecting groups is an ester.

9. The method of claim 8, wherein the ester selected from the group consisting of formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, p-P-phenylacetate, 3-phenylpropionate, 4-oxopentanoate (levulinate), 4,4-(ethylenedithio)pentanoate, pivaloate, adamantoate, crotonate, 4-methoxycrotonate, benzoate, p-phenylbenzoate, 2,4,6-trimethylbenzoate (mesitoate), 2-iodobenzoate, 4-azidobutyrate, 4-nitro-4-methylpentanoate, o-(dibromomethyl)benzoate, 4-(methylthiomethoxy)butyrate, 2-(methylthiomethoxymethyl)benzoate, 2,6-dichloro-4-methylphenoxyacetate, 2,6-dichloro-4-( 1,1,3,3-tetramethylbutyl)phenoxyacetate, 2,4-bis(1,1 -dimethylpropyl)phenoxyacetate, chlorodiphenylacetate, isobutyrate, monosuccinoate, (E)-2-methyl-2-butenoate (Tigloate), o-(methoxycarbonyl)benzoate, p-P-benzoate, α-naphthoate, dimethylphosphinothioyl, and 2,4-dinitrophenylsulfenate.

10. The method of claim 1, wherein the phenol protecting group is a carbonate.

11. The method of claim 10, wherein the carbonate is selected from the group consisting of methyl, 9-fluorenylmethyl, ethyl, 2,2,2-trichloroethyl, 2-(methylthiomethoxy)ethyl, 2-(trimethylsilyl)ethyl, 2-(phenylsulfonyl)ethyl, 2-(triphenylphosphonio)ethyl, isobutyl, vinyl, allyl, p-nitropheny, benzyl, p-methoxybenzyl, 3,4-dimethoxybenzyl, o-nitrobenzyl, p-nitrobenzyl, S-benzyl thiocarbonate, 4-ethoxy-1-naphthyl, and methyl dithiocarbonate.

12. The method of claim 1, wherein the phenol protecting group is selected from the group consisting sulfate, methanesulfonate (mesylate), benzylsulfonate, tosylate, 2-formylbenzenesulfonate, nitrate, borate, alkyl N,N,N′,N′-tetramethylphosphorodiamidate, and N-phenylcarbamate.

13. The method of claim 1, wherein the at least one organic solvent in step (b) is selected from the group consisting of methanol, ethanol, acetone, methyl ethylketone, isopropyl alcohol, acetic acid, water, ethyl acetate, ethylene dichloride, dichloromethane, and mixtures thereof.

14. The method of claim 1, wherein the solution of step (b) is heated to a temperature above room temperature.

15. The method of claim 14, further comprising cooling the solution of step (b) to room temperature or below and evaporating the at least one organic solvent to induce recrystallization of curcumin.

16. The method of claim 1, wherein the solution of step (b) is placed in a refrigerator to assist the recrystallization of curcumin.

17. The method of claim 1, further comprising the step of collecting the crystals of pure curcumin.

18. The method of claim 17, wherein the collecting step comprises vacuum filtration.

19. The method of claim 17, further comprising the step of drying the crystals of pure curcumin.
